(19) United States
(12) Patent Application Publication
(10) Pub. No.: US 2013/0338604 A1
(43) Pub. Date: Dec. 19, 2013

(54) CLOSURE AND METHOD FOR PRODUCING A CLOSURE

(71) Applicant: Tilman Roedle, Wolfegg (DE)

(72) Inventor: Tilman Roedle, Wolfegg (DE)

(73) Assignee: VETTER PHARMA-FERTIGUNG GMBH & CO. KG, Ravensburg (DE)

(21) Appl. No.: 14/002,535

(22) PCT Filed: Feb. 23, 2012
(86) PCT No.: PCT/EP2012/000786

§ 371 Date: Aug. 30, 2013

(30) Foreign Application Priority Data
Mar. 3, 2011 (DE) 10 2011 013 791.2
Jul. 15, 2011 (DE) 10 2011 107 276.8
Aug. 24, 2011 (DE) 10 2011 111 552.1

(51) Int. Cl. A61M 5/31; A61J 1/14

(52) U.S. Cl. CPC A61M 5/3134; A61J 1/1412
USPC 604/199; 604/263; 264/250

(57) ABSTRACT

A closure for a syringe or carpule includes a main body and a sealing element for engaging in a sealing manner with an opening of the syringe or carpule when the closure is arranged in its sealing position on the syringe or carpule. The closure is embodied in a single piece.

The invention relates to a closure according to the preamble of claim 1, a closure according to the preamble of claim 3 and a method for manufacturing a closure according to the preamble of claim 14.

Closures of the type being discussed here and methods for manufacturing them are known. Such a closure for a syringe or carpule has a main body and a sealing element that is embodied and/or arranged such that it engages in a sealing manner with an opening of a syringe or carpule when the closure is arranged in its sealing position on the syringe or carpule. Closures are also known that have a duct that passes through the main body and the sealing element and has a proximal and a distal end. In this case, a sealing cap for sealing the distal end of the duct is provided, as well as a safety cap that engages around the sealing cap. The main body and the sealing element on the one hand and the sealing cap and the safety cap on the other hand are embodied in two pieces. The closures must therefore be preassembled before they can be placed onto a syringe or carpule. The individual parts are typically quite small and are therefore easily lost, particularly because they cannot be handled as a unit. Moreover, the manufacture of the individual parts requires separate production steps and considerable logistical effort for storage and allocation.

It is therefore the object of the invention to provide a closure and a method for manufacturing same wherein the cited drawbacks do not occur.

The object is achieved through the provision of a closure with the features of claim 1. As already noted, it has a main body and a sealing element. It is characterized in that it is embodied in a single piece. As a result, preassembly is omitted, and the individual elements of the closure cannot be lost. This, in turn, reduces storage costs and logistical effort.

Preferably, the closure is manufactured using the two-component injection molding method as a one-piece element. This, in particular, enables the injection molding of the main body from a first, comparatively hard material and the sealing element from a second, comparatively soft or elastic material. A production step is eliminated because the two elements need not be manufactured in separate processes.

The object is also achieved through the provision of a closure with the features of claim 3. In addition to the main body and the sealing element, it also has a duct that passes through the main body and the sealing element and has a proximal and a distal end. It also comprises a sealing cap for sealing the distal end of the duct, as well as a safety cap that engages around the sealing cap. The closure is characterized in that either the main body and the sealing element or the sealing cap and the safety cap or both pairs of parts are embodied in a single piece. The main body can therefore be embodied in a single piece with the sealing element. It is also possible for the sealing cap and the safety cap to be embodied in a single piece. Furthermore, it is possible for both the main body and the sealing element as well as the sealing cap and the safety cap to each be embodied as a single piece. Finally, the entire closure can also be preferably embodied in a single piece. The previously cited advantages are achieved; in particular, the preassembly is eliminated at least with respect to the parts embodied in a single piece. Production, storage and logistical costs are reduced. Moreover, the loss of small elements is reliably avoided.

Preferably, the main body and the sealing element and/or die sealing cap and the safety cap are manufactured as one-piece elements using the two-component injection molding method. It is also possible to manufacture the entire closure as a one-piece element using the two-component injection molding method. This makes it possible, in particular, to manufacture the main body and/or the safety cap from a comparatively hard material and the sealing element and/or the sealing cap from a comparatively soft or elastic material. The main body and the safety cap are intended, namely, to lend stability to the closure, whereas the sealing element and the sealing cap are intended to close the syringe or carpule and the distal end of the duct in a sealing manner, for which purpose they preferably have a certain elasticity to ensure that they seat tightly.

Preferably, a closure in which the elements joined together using the two-component injection molding method has—when seen in the radial direction—at least one projection and at least one recess which engage in one another in a locking manner. This results in an optimum connection of the elements. At the same time, rotation is prevented. It has been shown that it is especially favorable if a sealing element cannot rotate relative to a supporting element. In this way, leaks can be prevented that might occur as a result of the relative rotation.

Also preferred is a closure in which at least one radial projection of an element—when seen in the axial direction—is engaged around on both sides by walls of a radial recess of the other element, with the projection engaging into the recess. In this case, axial forces can be transferred from one element to the other element. Particularly, one element is carried along when the other element is removed from a syringe or carpule, because axial forces can be introduced via the at least one projection and the walls engaging around same from one element to the other.

Additional preferred embodiments follow from the subclaims.

The object is also achieved through the provision of a method for the manufacture of a closure with the features of claim 14. The method is characterized by the following steps: First, a main body and/or a safety cap is injected from a first material. Then, a sealing element is injection molded onto the main body and/or a sealing cap is injection molded onto the safety cap. A second material is used here. A two-component injection molding method is therefore used to manufacture the closure. In doing so, the first material is preferably comparatively hard, whereas the second material is comparatively soft or elastic. As a result, the first material is suited to forming a supporting body, whereas the second material is suited to forming a part that has sealing characteristics.

A method is preferred in which both injection molding steps are performed in the same tool. This saves additional production steps.

Especially preferred is a method in which the elements produced in the two injection molding steps are locked together during injection molding.

Embodied on the elements are at least one projection and at least one recess, which engage in one another. In particular, a relative rotation of the elements that might lead to leaks can be prevented in this way.

Additional advantageous embodiments follow from the subclaims.

In the following, the invention is explained in further detail on the basis of the figures.

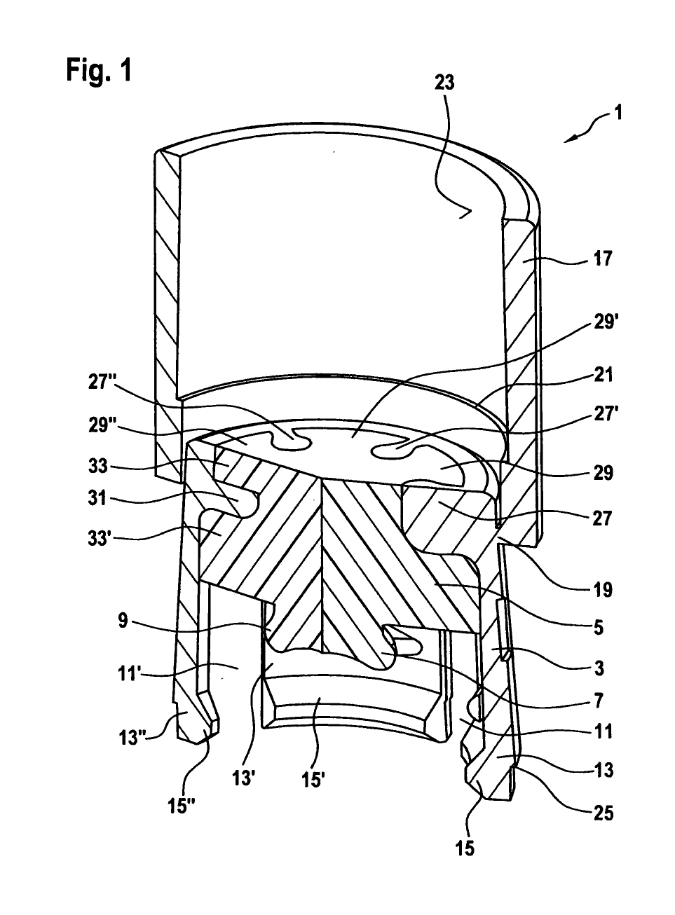

FIG. 1 shows a first sample embodiment of a closure in an isometric sectional view in a state before the closure is placed onto a syringe or carpule;

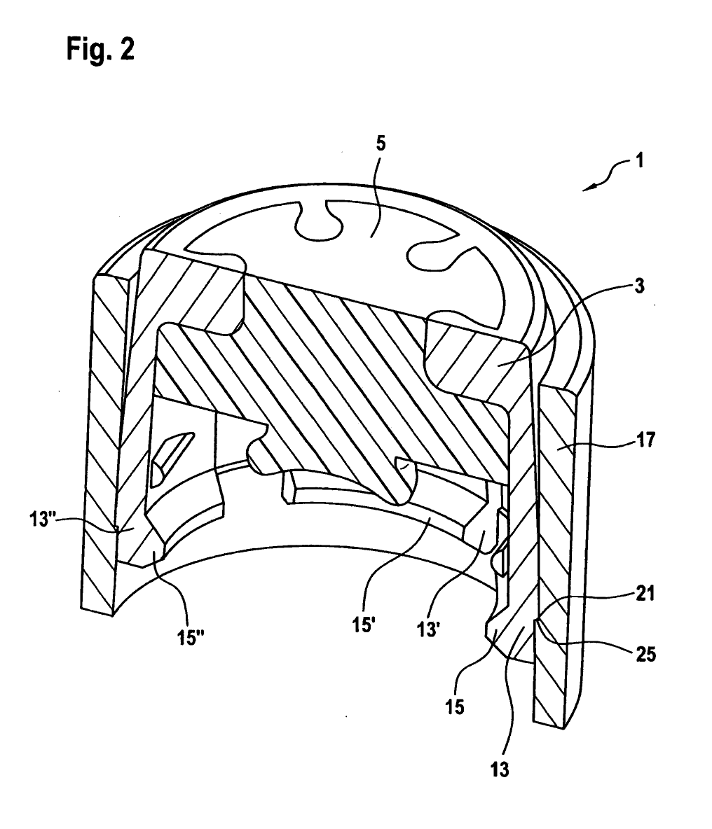

FIG. 2 shows another isometric sectional view of the sample embodiment according to FIG. 1 in a state in which the closure is placed on a syringe or carpule (not shown), and

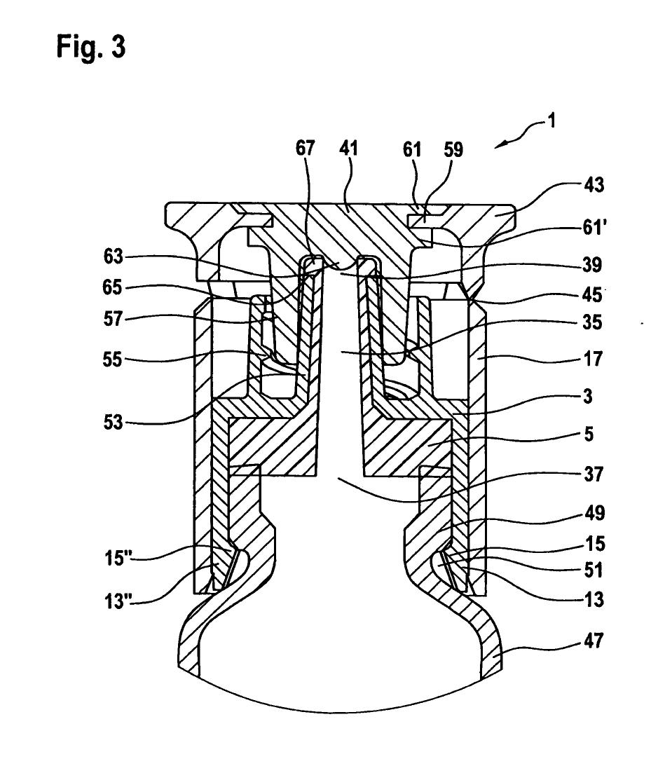

FIG. 3 shows another sample embodiment of a closure that is placed onto a syringe or carpule.

FIG. 1 shows an isometric sectional view with angled sectional plane of a first sample embodiment of a closure 1. It is intended for the sealing of a syringe or carpule. It is also possible to seal a dual chamber system using the closure 1. The closure has a main body 3 and a sealing element 5.

The sealing element 5 is embodied and/or arranged such that it engages in a sealing manner with an opening of a syringe or carpule, optionally of a dual chamber system as well, when the closure is arranged in its sealing position on the syringe or carpule or the dual chamber system. In particular, the sealing element 5 is comparatively elastic, so that it can be compressed at least slightly. In contrast, the main body 3 is comparatively hard. It serves as a supporting element for the sealing element 5. If the main body 3 is arranged with the sealing element 5 on a syringe or carpule in its sealing position, it preferably locks onto the syringe or carpule in such a way that axial forces are introduced into the sealing element 5 which compress it. It then lies against the opening of the syringe or carpule in a sealing manner.

The sealing element 5 has a central projection 7 that can engage in the opening of a syringe or carpule. Arranged on this is—when seen in the circumferential direction—a radial, extended flaring 9 whose external diameter is preferably larger than the internal diameter of an opening of the syringe or carpule that is sealed with the closure 1. Accordingly, the flaring 9 is also compressed when the closure 1 is brought into its sealing position. It then lies against the inner wall of the opening in a sealing manner.

At least in an area that—when seen in the axial direction—is arranged beneath the sealing element 5, the main body 3 has at least one, here several—when seen in the circumferential direction—axial recesses arranged at an angular distance with respect to each other, two of which recesses 11, 11′ are shown. When seen in the circumferential direction, tabs are embodied between the recesses, three of which tabs 13, 13′, 13″ are shown here. On one end of the tabs 13, 13′, 13″ facing a syringe or carpule in the sealing position of the closure 1, these have projections 15, 15′, 15″ which extend inward when seen in the radial direction.

A syringe or carpule or a dual chamber system that is sealed with the closure 1 has in its opening area a flange on which is embodied an undercut or a groove into which the projections 15, 15′, 15″ engage in the sealing position of the closure 1. As a result of the recesses 11, 11′, the tabs 13, 13′, 13″ have a certain elasticity, so they are able to swing somewhat radially outward when the closure is put in place so that the closure 1 can be pushed over the flange. Once the projections 15, 15′, 15″ reach the undercut or groove, the tabs move elastically again radially inward, so that the projections 15, 15′, 15″ engage in the undercut or groove and hold the closure 1 on the flange.

In order to secure the closure 1 on the opening, a retaining ring 17 is provided. In the state shown in FIG. 1, in which the closure 1 is not yet arranged in its sealing position, this is preferably connected to the main body 3 via at least one tear-off 19. In this state, the retaining ring 17 does not engage around the tabs 13, 13′, 13″. After the closure has been brought into its sealing position, the retaining ring 17 is moved in the direction of the projections 15, 15′, 15″. It has an edge 21 which—when seen in the radial direction—protrudes inwardly. In doing so, it preferably extends over the entire periphery of the retaining ring 17 in the area of its inner surface 23. If the retaining ring 17 in FIG. 1 is moved downward, the edge 21 engages behind a corresponding (when seen in the radial direction), outwardly protruding edge 25 which—when seen in the circumferential direction—extends along the tabs 13, 13′, 13″. In this way, the retaining ring 17 is held in a locking position in which it—when seen in the axial direction—cannot be moved relative to the main body 3. At the same time, the tabs 13, 13′, 13″ can no longer swing radially outward, because they are engaged around and held by the retaining ring 17. Overall, the closure 1 is held securely and solidly in its sealing position in this way.

The closure 1 can preferably be used in connection with dual chamber systems in which substances are freeze-dried in a distal chamber. In this case, the flange of such a dual chamber systems has an additional groove into which the projections 15, 15′, 15″ can engage without the sealing element 5 lying against the opening of the dual chamber systems in a sealing manner. An upper locking position of the closure 1 is practically realized in this way. A fluid path is then formed from the interior of the distal chamber via the recesses 11, 11′ into the vicinity of the dual chamber systems. Solvent can evaporate through this until freeze-dried product is left behind. After freeze-drying, the closure 1 can be brought into its lower locking position in which the projections 15, 15′, 15″ engage in the undercut or first groove, which was already explained. The dual chamber system is then tightly sealed by the closure 1.

The closure 1 is embodied as one piece. This means that the main body 3 and the sealing element 5 are not separate parts that are pieced together with the closure 1. Instead, they form a preferably inseparable unit.

Especially preferably, the closure is manufactured using the two-component injection molding method. In this way, it can be manufactured as a one-piece element.

The main body preferably comprises a thermoplastic polymer, preferably polypropylene; especially preferably, it is comprised of this material. Polypropylene is a comparatively hard plastic that is well-suited to the formation of the main body 3 as a supporting body for the sealing element 5.

This is preferably manufactured from a material which comprises TPE (thermoplastic elastomer). Especially preferably, the sealing element 5 is comprised of TPE. This is a comparatively elastic material, which is very well suited to the sealing characteristics of the sealing element 5. Moreover, TPE is a co-called material suitable for primary contact that may come into contact with medical agents. For this reason, a surface that can come into direct contact with an agent can comprise TPE in any case or be comprised of TPE.

Polypropylene, for example, is not suitable for primary contact. Consequently, the closure 1 is preferably embodied such that only such elements which comprise TPE, preferably which are comprised of TPE, come into contact with an agent that is arranged in a chamber facing the closure 1 a syringe or carpule [sic] or, optionally, in the distal chamber of a dual chamber system.

The retaining ring 17 is preferably manufactured together with the main body 3 and, especially preferably, from the same material as it. In a two-component injection molding method, the main body 3, the retaining ring 17 and preferably the tear-offs 19 as well as therefore manufactured in the same injection molding step. In this respect, the retaining ring 17 is then part of the main body 3.

The elements joined together using the two-component injection molding method preferably have—when seen in the radial direction—at least one projection and at least one recess, which engage in one another in a practically locking manner.

In the depicted sample embodiment, the two elements connected to one another, namely the main body 3 and the sealing element 5 have—when seen in the circumferential direction—several radial projections and recesses. Here, three projections 27, 27′, 27″ of the main body 3 are shown. These engage into corresponding recesses of the sealing element 5 which are not provided here with reference symbols. Provided between the projections 27, 27′, 27″ are—when seen in the circumferential direction—recesses of the main body 3 into which projections 29, 29′, 29″ of the sealing element 5 engage. In this way, the main body 3 and the sealing element 5 are practically locked together. In particular, a relative rotation between the two elements, which may otherwise lead to a leak in the contact area of the sealing element 5 to the carpule or syringe and/or in the contact area of the sealing element 5 to the main body 3, is thus prevented.

Also provided on the main body 3 is at least one radial projection projection 31 which—when seen in the axial direction—is engaged around on both sides by walls 33, 33′ of a radial recess of the sealing element 5. The projection 31 engages here into the corresponding recess. In addition or alternatively, a provision can be made that a radial projection of the sealing element 5 is engaged around—when seen in the axial direction—on both sides of walls of a radial recess of the main body 3, and the projection of the sealing element 5 then engages into the recess of the main body 3. In general, one of the elements joined together using the injection molding method preferably has a radial projection which—when seen in the axial direction—is engaged around on both sides by walls of at least one radial recess of the other element, and the at least one projection engages in the at least one recess.

As a result, it is possible to introduce axial forces from the main body 3, for example, into the sealing element 5.

FIG. 2 shows a view of the sample embodiment of a closure 1 according to FIG. 1 in the sealing position. Same and functionally equal elements are provided with the same reference symbols, so reference is made in this respect to the preceding description. The retaining ring 17 is moved here into its lower position. Here, the edge 21 engages behind the edge 25, thus preventing movement of the retaining rings 17 back into its upper position. At the same time, the retaining ring 17 prevents the tabs 13, 13′, 13″ from swinging out radially, so that the projections 15, 15′, 15″ engage securely into the undercut or groove on the flange of the syringe or carpule. The closure 1 is securely held in its sealing position in this way. Finally, it is hardly possible any longer in the depicted sample embodiment to remove the closure 1 from the syringe or carpule without destruction or to move it out of its sealing position.

The sealing element 5 is embodied here as a septum that can preferably be penetrated by a cannula. The illustrated closure 1 is therefore especially suited to the sealing of carpules. These are preferably used in pens and auto-injectors.

FIG. 3 shows another sample embodiment of a closure 1. Same and functionally equal elements are provided with the same reference symbols, so reference is made in this respect to the preceding description. The sample embodiment depicted here is preferably suitable for use with syringes or dual chamber systems. The closure 1 has a duct 35 that passes through the main body 3 and the sealing element 5. It has a proximal end 37 and a distal end 39. The closure further comprises a sealing cap 41 that seals the distal end 39. What is more, a safety cap 43 is provided which engages around the sealing cap 41. It is connected to the retaining ring 17 via at least one tear-off 45. In the sample embodiment shown in FIG. 3, this has a somewhat different geometry than in the sample embodiment according to FIGS. 1 and 2, but it fulfills the same function.

FIG. 3 also shows the distal end of a syringe 47. In particular, an opening area 49 and an undercut or groove 51 are shown. As was described in connection with the sample embodiments according to FIGS. 1 and 2, in the sealing position of the closure 1, the projections of the main body 3, of which the projections 15, 15″ are illustrated here, engage in the undercut or groove 51, thus holding the closure 1 in its sealing position. The retaining ring 17 prevents the tabs, of which the tabs 13, 13″ are illustrated here, from swinging radially outward, thus securely holding the closure 1 in its sealing position.

In the depicted sample embodiment, the main body 3 has a neck 53 for coupling with a dispensing element for an agent, for example a cannula or syringe needle. Preferably, the neck 53 is cone-shaped. Especially preferably, it is embodied as a Luer cone.

Preferably, a Luer thread 55 is also provided on the main body 3 which engages around the neck 53 and is used to couple the main body with a dispensing element in an inherently known manner.

The sealing cap 41 preferably lies with a wall area 57 in a sealing manner against the neck 53. Preferably, the safety cap 43 and the sealing cap 41 are embodied as a one-piece element. Especially preferably, these elements are manufactured as a one-piece element using a two-component injection molding method.

Alternatively or at the same time, it is preferred that the main body 3 and the sealing element 5 be embodied as a one-piece element and especially preferably manufactured as a one-piece element using the two-component injection molding method.

It is also possible for the closure 1 to be embodied in its entirety as a single piece and preferably manufactured using the two-component injection molding method as a one-piece element.

Particularly in the safety cap 43 and the sealing cap 41, at least one projection and at least one recess are preferably provided which engage in each other in a practically locking manner. Especially preferably, several radial projections and recesses—when seen in the circumferential direction—are provided. As shown in FIG. 3, either an extending—when seen in the circumferential direction—annular projection 59 or at least two mutually opposing projections of the safety cap 43 engage here in at least one annular recess or at least two opposing recesses of the sealing cap 41. Both designs, which cannot be distinguished in the sectional illustration of FIG. 3, are possible. It is also possible to provide—when seen in the circumferential direction—several projections and recesses at an angular distance from one another. It is also possible for the projections on the sealing cap 41 and the recesses to be embodied accordingly on the safety cap 43. In any case, the one projection or the several projections—when seen in the axial direction—are engaged around on both sides by walls, here walls 61, 61′ for the sake of example, of the corresponding recess. In doing so, the projection 59 engages in the corresponding recess. In this way, axial forces, for example from the safety cap 43, can be transferred into the sealing cap 41. If the safety cap 43 is removed from the closure 1, the sealing cap 41 is taken along at the same time in this way. It is therefore removed at the same time together with the safety cap 43. This is especially favorable because an additional step for the activation of the syringe 47 can be eliminated in this way.

In the sample embodiment shown in FIG. 3 as well, the main body 3 preferably has a thermoplastic polymer, preferably polypropylene, or is especially preferably comprised thereof. Likewise, the safety cap 43 preferably comprises a thermoplastic propylene, preferably polypropylene, or is preferably comprised thereof.

The sealing element 5 preferably comprises TPE or is especially preferably comprised thereof. Likewise, the sealing cap 41 preferably comprises TPE or is especially preferably comprised thereof. The sealing cap 41 is preferably embodied from a comparatively soft or flexible material so that it can lie with the wall area 57 in a sealing manner against the neck 53, thus sealing the distal end 39 of the duct 35.

For this purpose a central projection 63 is provided on the sealing cap 41 which central projection 63 lies, on the one hand, in a sealing manner against the distal end 39 and, on the other hand, protrudes at least in areas into the duct 35.

In one sample embodiment of the closure 1, the sealing element 5 protrudes into the duct 35 but does not penetrate completely through it. In this case, the projection 63 protrudes at least far enough into the duct 35 that it lies in a sealing manner against the sealing element 5. Preferably, both the sealing element 5 and the sealing cap 41 comprise material that is suitable for primary contact or are especially preferably comprised of such material. As a result of the sealing contact of the projection with the sealing element 5, a substance that is disposed in the syringe 47 is reliably prevented from coming into contact with the main body 3. The main body 3 can then comprise a material that is not suitable for primary contact.

In the depicted sample embodiment, the sealing element 5 protrudes completely through the duct 35 in the main body 3. In particular, the sealing element 5 engages completely around the neck 53. In an especially preferred manner, an annular contact surface 67 is formed on a distal end 65 of the neck 53, which annular contact surface 67 lies against the distal end 65 of the neck 53 and covers same. Consequently, the projection 63 can be comparatively short in the depicted sample embodiment because it lies tightly against the contact surface 67. It this way as well, it is ensured that none of the substance present in the syringe 47 can come into contact with the material of the main body 3, because the sealing element 5 engages completely around the duct 35 and particularly the neck 53 and forms the contact surface 67.

The spatial and geometric arrangement shown in FIG. 3 of the main body 3 and of the sealing element 5 protruding through it can be manufactured in an especially advantageous manner using the two-component injection molding method. Here, a sealing element 5 can readily be injection molded on the main body that protrudes completely through the duct 35 and particularly the neck 53, especially preferably forming a contact surface 67.

For this reason, in the sample embodiment depicted in FIG. 3, the main body 3 and the sealing element 5 are especially preferably embodied as a one-piece element and preferably manufactured using the two-component injection molding method.

Alternatively or at the same time, the sealing cap 41 and the safety cap 43 are embodied as a one-piece element and preferably manufactured using the two-component injection molding method. In this way, the at least one projection 59 and the at least one corresponding recess can particularly readily be provided with the walls 61, 61′. The sealing cap 41 is then advantageously taken along as well upon removal of the safety cap 43.

Especially preferably, in the sample embodiment according to FIG. 3 as well, the main body 3 and/or the safety cap 43 comprise(s) polypropylene or is/are comprised thereof. Polypropylene is a relatively hard material that is not suitable for primary contact. The sealing element 5 and/or the sealing cap 41 preferably comprise(s) TPE or is/are especially preferably comprised thereof. TPE is a relatively elastic material that is suitable for primary contact. As an especially preferred material, the relatively elastic elements, which is to say, in particular, the sealing element 5 and/or the sealing cap 41, can comprise TPE-V (crosslinked thermoplastic elastomer) or be comprised thereof.

The method for manufacturing the closure using a two-component injection molding process preferably comprises the following steps:

First, the main body 3 is injection molded from a first material. At the same time, or in another step, the safety cap 43 is optionally and preferably injection molded from the same material. In particular, if a retaining ring 17 is provided on the main body 3 to which the safety cap 43 is optionally connected, the main body 3, the retaining ring 17 and, optionally, the safety cap 43 are injected together from the first material. It is also possible for these process steps to be separated.

After that, the sealing element 5 is injection molded onto the main body 3. A second material is preferably used here. At the same time or in a separate step—if the sealing cap 41 and the safety cap 43 are provided in the closure 1—the sealing cap 41 is injection molded onto the safety cap 43. Here, too, a second material is used.

Especially preferably, the main body 3 and the sealing element 5 are injection molded in the same tool. Likewise, the safety cap 43 and the sealing cap 41 are preferably injection molded in the same tool, especially preferably in the same tool as the main body 3 and the sealing element 5.

As already indicated, the main body 3 is preferably injection molded from a material which comprises a thermoplastic polymer, particularly polypropylene, and is preferably comprised of a thermoplastic polymer, preferably polypropylene. The same applies to the safety cap 43. The sealing element 5 is preferably injection molded from a material which comprises TPE and is preferably comprised of TPE. The same applies to the sealing cap 41.

Especially preferably, the main body 3 and the sealing element 5 are locked together during injection molding. This suggests that at least one projection or at least one recess be provided on each of the elements that engage in each other in a locking manner. The sealing cap 41 and the safety cap 43 are also preferably locked together in an appropriate manner during injection molding.

In the sample embodiment according to FIG. 3, the following is also shown: As a result of the safety cap 43 and the sealing cap 41 being manufactured as a one-piece element using a two-component injection molding method, and particularly as a result of the two elements being locked together, the overall height of the closure 1 can be reduced. Namely, if the sealing cap 41 and the safety cap 43 are embodied in two pieces, the safety cap 43 must not only engage around the sealing cap 41 but also engage over it in order to protect the sealing cap 41 from loss or unauthorized removal. In contrast, if the sealing cap 41 is embodied in a single piece with the safety cap 43, particularly locked together with same, it is sufficient if the safety cap 43 engages around the sealing cap 41 as is shown in FIG. 3. The closure 1—when seen in the longitudinal direction—can therefore have a shorter extension or smaller total height.

Overall, it can be seen that, with the aid of the method proposed here, closures can be manufactured in which an optimal connection of two elements, for example of main body 3 and sealing element 5 but also safety cap 43 and sealing cap 41 is ensured. Preassembly is unnecessary, because the elements are provided in a single piece. No small parts can be lost. At the same time, it is possible to prevent rotation through the locking of the two elements. Overall, with the aid of the proposed closures and the proposed method, production, storage and logistical costs can be reduced.

1-18. (canceled)

19. A closure for a syringe or carpule, the closure comprising:
a main body; and
a sealing element for engaging in a sealing manner with an opening of the syringe or carpule when the closure is arranged in a sealing position on the syringe or carpule;
wherein the closure is embodied in a single piece.

20. The closure as set forth in claim 19, wherein the closure is manufactured as a one-piece element using a two-component injection molding method.

21. The closure as set forth in claim 19, further comprising:
a duct protruding through the main body and the sealing element and having a proximal and a distal end;
a sealing cap sealing the distal end of the duct; and
a safety cap which engages around the sealing cap;
wherein the main body and the sealing element and/or the sealing cap and the safety cap are embodied in a single piece.

22. The closure as set forth in claim 21, wherein the main body and the sealing element and/or the sealing cap and the safety cap are manufactured as a one-piece element or as one-piece elements using a two-component injection molding method.

23. The closure as set forth in claim 21, wherein at least one of the main body and the safety cap comprises a thermoplastic polymer.

24. The closure as set forth in claim 21, wherein at least one of the sealing element and the sealing cap is at least partially constructed of TPE.

25. The closure as set forth in claim 22, wherein the one-piece element includes at least one projection and at least one recess that are engaged in a locking manner.

26. The closure as set forth in claim 22, wherein the one-piece element includes a plurality of radial projections and a plurality of recesses engaged in a locking manner.

27. The closure as set forth in claim 25, wherein the at least one radial projection of an element is engaged around on both sides by walls of a radial recess of the other element, with the projection engaging in the recess.

28. The closure as set forth in claim 21, wherein the sealing element protrudes completely through the duct in the main body.

29. The closure as set forth in claim 19, wherein the main body has a neck for a cannula.

30. The closure as set forth in claim 29, wherein the neck is a Luer cone.

31. The closure as set forth in claim 29, wherein the sealing element protrudes completely through the neck.

32. The closure as set forth in claim 19, in combination with the syringe or carpule.

33. A method of manufacturing the closure of claim 19, the method comprising:
injection molding the main body from a first material; and
injection molding the sealing element onto the main body with a second material.

34. The method as set forth in claim 33, wherein both injection molding steps are performed in a same tool.

35. The method as set forth in claim 33, wherein at least one of the main body and the sealing cap is injection molded from a material which comprises polypropylene.

36. The method as set forth in claim 33, wherein at least one of the sealing element and the sealing cap is injection molded from a material which comprises TPE.

37. The method as set forth in claim 33, wherein injection molding the sealing element includes locking the sealing element to the main body.

38. A method of manufacturing the closure of claim 21, the method comprising:
injection molding at least one of the main body and the safety cap of a first material; and
injection molding the sealing element with a second material and onto at least one of the main body and the sealing cap.
